(19) United States
(12) Patent Application Publication
(10) Pub. No.: US 2023/0114385 A1
(43) Pub. Date: Apr. 13, 2023

(54) MRI-BASED AUGMENTED REALITY ASSISTED REAL-TIME SURGERY SIMULATION AND NAVIGATION

(71) Applicant: City University of Hong Kong, Hong Kong (HK)

(72) Inventors: Camroo AHMED, Hong Kong (HK); Sesugh Samuel NDER, Hong Kong (HK); Lai Hang Leanne CHAN, Hong Kong (HK); Kening ZHU, Hong Kong (HK); Yat Ming Peter WOO, Hong Kong (HK)

(21) Appl. No.: 17/961,577

(22) Filed: Oct. 7, 2022

Related U.S. Application Data
(60) Provisional application No. 63/253,557, filed on Oct. 8, 2021.

(51) Int. Cl. A61B 34/20; A61B 90/00

(52) U.S. Cl. CPC A61B 34/20; A61B 90/36; A61B 2090/365; A61B 2034/2051; A61B 2034/2055

(57) ABSTRACT

An MRI-based surgical navigation method of providing a personalized augmented reality of targeted internal organs with real-time intraoperative tracking is provided. Briefly, two-dimensional MRI images of targeted internal organs are segmented into a plurality of segmented data and recombined thereof to generate a three-dimensional volumetric model of the targeted internal organs. An augmented reality-based three-dimensional simulation model including anatomical features and spatial information of the targeted internal organs is obtained to be overlaid with the three-dimensional volumetric model while collecting real-time feedback of surgical operations. The anatomical features and spatial information data of the targeted internal organs are processed to generate robust and accurate navigation coordinates, which will be outputted to an augmented reality-based three-dimensional simulation and real-time anatomical model capture and display device for assisting medical practitioners to visualize surgical paths and specific anatomical features of an individual receiving said surgical operations.

### CROSS-REFERENCE OF RELATED APPLICATION

This present application claims the benefit of U.S. Provisional Patent Application No. 63/253,557 filed Oct. 8, 2021, which is incorporated by reference herein in its entirety.

### FIELD OF THE INVENTION

The present invention relates to augmented reality application fields. Particularly, it relates to a method and system for real-time tracking and visualizing targeted internal organs during pre-operative planning and intraoperative stages of surgical operation by integrating augmented reality technologies.

### BACKGROUND OF THE INVENTION

Some internal organ surgeries are difficult to perform as the operation is confined. Taking brain surgery as an example, it is hard to expose or visualize the entire brain for neurosurgeons to make a medical plan or an operation strategy. Using augmented reality (AR) technology to visualize organs is an option for surgeons to visualize the brain with minimal invasiveness and high safety. However, only a few AR systems are practiced in the clinical fields. Currently, commercially available neurosurgery navigation systems only provide 2D image guidance, so that surgeons have to mentally visualize the 3D structure of brain. During surgeries, the surgeons need to control the surgical field instruments while looking at an external display at the same time. It takes extra effort to do a high-precision operation, like brain surgery, if the surgeons cannot look at the operation site. Therefore, a real-time 3D view of brain anatomy can help surgeons to better visualize the operation

With immersive AR technology and computer vision, a computer-generated 3D brain anatomical model can be overlaid onto the surgeon's vision simultaneously with the view of the surgical field. The use of AR technology in the field of healthcare has been growing rapidly. Currently available head mounted AR systems have promising features in visualizing 3D anatomical models. However prior art AR applications for surgery fail to include real-time tracking for surgical navigation.

Therefore, a non-human intervention, personalized and real-time augmented reality-based three-dimensional visualization method of targeted internal organs is urgently needed in the field of complex surgeries including minimal invasive neurosurgery and spine surgery. The present invention addresses this need.

### DESCRIPTION OF RELATED ART

U.S. Pat. No. 9,646,423 describes systems and methods for providing AR in minimally invasive surgery including capturing pre-operative image data of internal organs of a patient, capturing intra-operative image data of the internal organs with an endoscope during a surgical procedure, registering the pre-operative image data and the intra-operative data in real time during the surgical procedure, tracking the position and orientation of the endoscope during the surgical procedure, and augmenting the intra-operative image data captured by the endoscope in real time with a rendering of at least a portion of an internal organ of the patient that is in registration with the real time intra-operative image data from the endoscope but outside of the field of view of the endoscope. This patent focuses on endoscopic imaging based intra-operative augmentation of image data. It only discloses a method to display an AR image by augmenting image captured by endoscope whereas the present invention focuses on pre-operative MRI based 3D volumetric reconstruction and intra-operative tracking to avoid the real-time reconstruction delay.

WO2017066373A1 provides an AR surgical navigation method including preparing a multi-dimensional virtual model associated with a patient. The method further includes receiving tracking information indicative of a surgeon's current view of the patient, including the surgeon's position relative to the patient and the surgeon's angle of view of the patient; identifying in the virtual model a virtual view based on the received tracking information, wherein the identified virtual view corresponds to the surgeon's view of the patient. The method further includes rendering a virtual image from the virtual model based on the identified virtual view; communicating the rendered virtual image to a display where the rendered virtual image is combined with the surgeon's view to form an AR view of the patient. This application is more focused on displaying an AR image that is correspondingly matched with the user's eye field to form an AR view of the patient, rather than MRI data for virtual volumetric 3D reconstruction and real-time intra-operative tracking.

U.S. Pat. No. 10,326,975 provides a real-time surgery method and apparatus for displaying a stereoscopic augmented view of a patient from a static or dynamic viewpoint of the surgeon, which employs real-time three-dimensional surface reconstruction for preoperative and intraoperative image registration. Stereoscopic cameras provide real-time images of the scene including the patient. A stereoscopic video display is used by the surgeon, who sees a graphical representation of the preoperative or intraoperative images blended with the video images in a stereoscopic manner through a see-through display. The patent does not specify the registration of the surgical field for intra-operative conditions which is necessary to provide guidance during surgery.

U.S. Pat. No. 7,493,153 provides a guide system for use by a user who performs an operation in a defined three-dimensional region, including a data processing apparatus for generating images of the subject of the operation in co-registration with the subject, a display for displaying the images to the user, a probe having a position which is visible to the user, and a tracking unit for tracking the location of the probe by the system and transmitting that location to the data processing apparatus, the data processing apparatus being arranged, upon the user moving the probe to a selection region outside and surrounding the defined region, to generate one or more virtual buttons, each of the buttons being associated with a corresponding instruction to the system, the data processing apparatus being arranged to register a selection by the user of any of the virtual buttons, the selection including positioning of the probe in relation to the apparent position of that virtual button, and to modify the computer-generated image based on the selection. This patent relates to AR-based system design and to the user interface for virtual models, focusing, in particular, on the probe position.

US20120113140 provides an AR system comprising a user-interaction region, a camera that captures images of an object in the user-interaction region, and a partially transparent display device which combines a virtual environment with a view of the user-interaction region, so that both are visible at the same time to a user. A processor receives the images, tracks the object's movement, calculates a corresponding movement within the virtual environment, and updates the virtual environment based on the corresponding movement. In another example, a method of direct interaction in an augmented reality system comprises generating a virtual representation of the object having the corresponding movement, and updating the virtual environment so that the virtual representation interacts with virtual objects in the virtual environment. From the user's perspective, the object directly interacts with the virtual objects. This patent focuses on the user interface of said AR systems without teaching an end-to-end surgical tracking system for pre- and intra-operative conditions.

### SUMMARY OF THE INVENTION

The present invention provides a novel AR-based system employing an AR-assisted real-time tracking function. Surgical tracking in an AR-based platform is a novel approach compared with the currently available 2D image guide display, which provides 6 DoF and, consequently, better visualization. As seen in the discussion of the related art above, a surgical navigation system combining AR with real-time tracking has not been used.

The present invention provides a means of integrating AR technology with a real-time tracking system with mainstream surgical technology to solve the drawbacks in the existing technology.

There are three main objectives of the present invention: (1) improving visualization technique with a novel 3D anatomical model reconstruction algorithm using deep learning from patient-specific MRI; (2) simulating the surgical procedure using interactive anatomical AR models; (3) developing a real-time surgical tracking system with the patient specific anatomical 3D AR models as an intra-operative solution which can provide complementary vision.

In accordance of a first aspect of the present invention, an MRI-based surgical navigation method of providing a personalized augmented reality of targeted internal organs of a subject with real-time intraoperative tracking is provided.

A plurality of two-dimensional MRI images of targeted internal organs is obtained by an MRI device. These images are segmented into a plurality of segmented data and then recombined to generate a three-dimensional volumetric model of the targeted internal organs. Further, an augmented reality-based three-dimensional simulation is provided to obtain an augmented reality-based three-dimensional simulation model including anatomical features and spatial information of the targeted internal organs. The augmented reality-based three-dimensional simulation model is overlaid with the three-dimensional volumetric model of the targeted internal organs while collecting real-time feedback of one or more surgical operations carried out on the targeted internal organs. The anatomical features and spatial information data of the targeted internal organs are processed to generate a plurality of robust and accurate navigation coordinates, which will be output to an augmented reality-based three-dimensional simulation and real-time anatomical model capture and display device for assisting medical practitioners of the one or more surgical operations to visualize at least a surgical path and specific anatomical features of an individual receiving said surgical operation. For generating the plurality of robust and accurate navigation coordinates, a combined optical and electromagnetic tracking system is used. By recognizing the optical markers, the system tracks the optical markers of the targeted internal organs and generates a set of tracking data. The set of tracking data is fed to a filter that can transform the data point through a non-linear function to generate the coordinates.

In one embodiment, said segmenting and recombining are carried out by a deep neural network to generate the three-dimensional volumetric shape of the targeted human body part with unique identification of non-specific and specific anatomical features.

In one embodiment, the augmented reality-based three-dimensional simulation and real-time anatomical model capture and display device collects at least one body appearance feature of the subject from a user's direction of view, and the at least one body appearance feature is in registration with a human appearance characteristics image database.

In one embodiment, the plurality of robust and accurate navigation coordinates is calculated based on the plurality of two-dimensional magnetic resonance imaging images and the at least one body appearance feature of the subject.

In one embodiment, the filter is an unscented Kalman filter for transforming the data points through the non-linear function in order to obtain a deep learning-based data forecast model.

In one embodiment, the unscented Kalman filter is cascaded with a deep neural network for predicting the surgical path of the medical instrument in the targeted internal organs.

In one embodiment, the minimally invasive surgeries include minimally invasive neurosurgery and spine surgery.

A second aspect of the present invention provides a surgical navigation system for providing patient-specific and surgical environment-specific pre-operative planning and intraoperative navigation. The system includes a magnetic resonance imaging (MRI) device for capturing a plurality of two-dimensional images of targeted internal organs. A deep neural network segments the plurality of the two-dimensional images of the targeted human body part to obtain segmented data of the two-dimensional images and recombines the segmented data to generate a three-dimensional volumetric shape of the targeted internal organs.

A combined optical and electromagnetic tracking system acquires data of optical markers' location at the targeted internal organs and transforms the data points through a non-linear function. An augmented reality-based three-dimensional simulation and real-time anatomical model capture and display device creates a three-dimensional anatomical simulation model during a simulated surgical operation based on the three-dimensional volumetric shape of the targeted human body part, collecting body appearance features, gathering real-time feedback of one or more surgical operations, overlaying the three-dimensional volumetric shape of the targeted human body part with the three-dimensional anatomical simulation model, displaying a predicted surgical path of medical instrument obtained during a surgery simulation process and other information related to pre-operative planning and intra-operative navigation including navigation coordinates of medical instrument and specific anatomical features of an individual receiving the surgical operations.

In one embodiment, the augmented reality-based three-dimensional simulation and real-time anatomical model capture and display device collects at least one body appearance feature of the subject from a user's direction of view, wherein the at least one body appearance feature is in registration with a human appearance characteristics image database.

In one embodiment, the combined optical and electromagnetic tracking system processes data of the anatomical features and spatial information of the targeted internal organs to generate a plurality of robust and accurate navigation coordinates.

In one embodiment, the plurality of robust and accurate navigation coordinates is calculated based on the plurality of two-dimensional magnetic resonance imaging images and the at least one body appearance feature of the subject

In one embodiment, the combined optical and electromagnetic tracking system comprises a filter for transforming the data points through the non-linear function.

In one embodiment, the filter is an unscented Kalman filter cascaded with the deep neural network for predicting the surgical path of the medical instrument in the targeted internal organs.

### BRIEF DESCRIPTION OF THE DRAWINGS

The patent application file contains at least one drawing executed in color. Copies of this patent application publication with color drawings will be provided by the Office upon request and payment of the necessary fee.

The above and other objects, features and advantages of the present invention will become more apparent to those of ordinary skill in the art by describing in detail exemplary embodiments thereof with reference to the accompanying drawings, in which:

FIGS. 1A-1C shows the transition from an MRI image to a volumetric model generated by a deep neural network; FIG. 1A depicts T1 MRI on Axial Plane; FIG. 1B depicts a deep learning-based Segmentation Mask; and FIG. 1C shows a 3D structure of the anatomy from the MRI;

FIGS. 2A-2B exhibit a 3D holographic superimposition of anatomical AR model on a dummy head;

FIG. 3 depicts a workflow of the anatomical AR Visualization; and

FIG. 4 shows the AR-based system block diagram.

### DETAILED DESCRIPTION

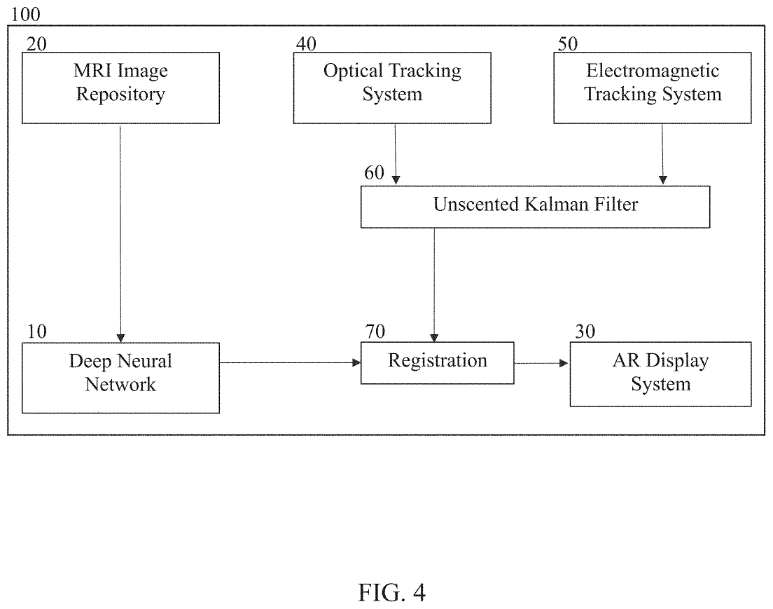

The MRI-based surgical navigation method and system for providing a personalized augmented reality of targeted internal organs of a subject with real-time intraoperative tracking is described in detail below. The invention is described in relation to neural surgery involving the brain; however, it is understood that the method and system are generally applicable to surgery in other parts of the body. Turning to the drawings in detail, FIG. 4 depicts a surgical system 100 according to an embodiment. System 100 includes deep neural network 10 and MRI image repository 20. In order to improve the visualization from 2D MRI images to a 3D volumetric shape, a deep neural network-based segmentation technique is applied by network 10 to every MRI image; segmented image parts are recombined to form a volumetric shape with unique identification of white matter, gray matter and any abnormalities. This deep neural network 10 employs a Bayesian Principle approach where a small portion of pretrained data is sufficient to train the entire network. The processed data is fed to registration 70 for matching with human appearance/organ characteristics image database. Segmentation and volume rendering of the anatomical model using such technique is a novel approach and this information is sent to an augmented reality display system.

The surgical system 100 further includes a head-mounted AR display system 30 which may be selected from a commercially available head mounted AR display systems. The surgical procedure is simulated on the 3D anatomical model displayed by display system 30 with real-time feedback of the surgical procedure. Surgical simulation using AR headsets is an example of the AR-based 3D simulation and real-time anatomical model capture and display device. Finally, the real-time tracking of surgical navigation using the AR anatomical 3D model which is designed based on the fusion of algorithms The surgical system uses an optical tracking system 40 and an electromagnetic tracking system 50 which feeds the information to Unscented Kalman filter 60 for transforming the data points through the non-linear function in order to obtain a robust and accurate navigation coordinate subsequently transmitted to registration 70. Combining both data, respectively processed by deep neural network 10 and unscented Kalman filter 60, and transforming them to AR display system 30, so that real time tracking information is included during the surgical simulation.

Tracking system 50 typically includes a surgical probe which may be mounted to a surgical instrument such as a catheter or be manually inserted into a surgical field. The surgical probe includes an image tracking element that provides images of anatomy in the vicinity of the probe. This imaging may be displayed as three images from three mutually orthogonal directions. Tracking of a surgical probe may be accomplished using electromagnetic, ultrasonic, mechanical, or optical techniques.

Functionalities of the system 100 are classified into three main principles: segmentation and 3D re-construction of anatomical model from patient-specific MRI scans; enhancing the anatomical visualization using Augmented Reality based 3D model; real-time tracking of surgical incision in the AR based anatomical 3D model. Also, three major applications of the present invention include: (1) preoperative planning, (2) surgery simulation and (3) intraoperative navigation.

(1) Preoperative planning system provides an in-depth 3D visualization of the anatomical model which is derived from the patient-specific MRI scans. The present system can help surgeons to set the trajectory of the surgical path. In conventional setup, this is done based on 2D MRI scans and involves human intervention to select suitable scans for re-construction into an 3D anatomical model.

(2) Surgical simulation based on the preoperative planning can provide the preliminary detail of the whole surgical process. These simulation results can help surgeons to deal with the unexpected which might arise during surgery. Most importantly, surgical simulation can help medical students and professionals to practice any specific surgical method repeatedly and conveniently.

(3) Intraoperative surgical system is the major focus of the present invention. The present system combines the tracking data from optical and electromagnetic tracking system to provide robust and accurate tracking data of surgical incision. Infused with the 3D anatomical model, this surgical incision tracking coordinates can give a clear picture of the whole surgery in an AR environment, significantly improving the safety of the whole operation.

In a preoperative condition, three-dimensional anatomical models are used as a guidance system to map the surgical procedures. Visualizing the anatomy and the related abnormalities in three-dimensions provides better accuracy than the conventional methods, hence, the quality of preplanning improves severalfold.

Further details relating to the construction and operation of surgical system 100 are discussed in connection with the Example, below.

### EXAMPLE

In order to create the three-dimensional anatomical model, open-source MRI datasets were employed. An augmented reality headset system 30, HoloLens2 (Microsoft Corporation) and an optical tracking system 40, OptiTrack V120 Trio (Natural Point, Inc. USA), were used in this example for tracking an optical marker's location and at the same time for creating the electromagnetic tracking system 50.

The data acquired from both tracking systems 40 and 50 are fed into an Unscented Kalman Filter 12 for a robust and accurate navigation coordinate which can be supplied to AR display system 30. The Unscented Kalman filter is a derivation from the original Kalman Filter which transforms the data points through a non-linear function for unscented transformation. As a result, the data points approximation is more correct and less prone to line-of-sight and magnetic field interruption errors. Data corrections and approximation using Unscented Kalman filter is a novel approach in this field. For better accuracy of data forecast, a deep learning-based data forecast model is in-line with the unscented Kalman filter.

The augmented reality headset 30 is used to visualize the tracking data that are supplied from registration 70 collecting the processed data from neural network 10 and unscented Kalman filter 60. Also, the hololens AR display system 30 is equipped with a depth camera system, allowing the system to collect the point cloud data of the patient's body appearance features and register it with a human appearance characteristics image database for holographic superimposition.

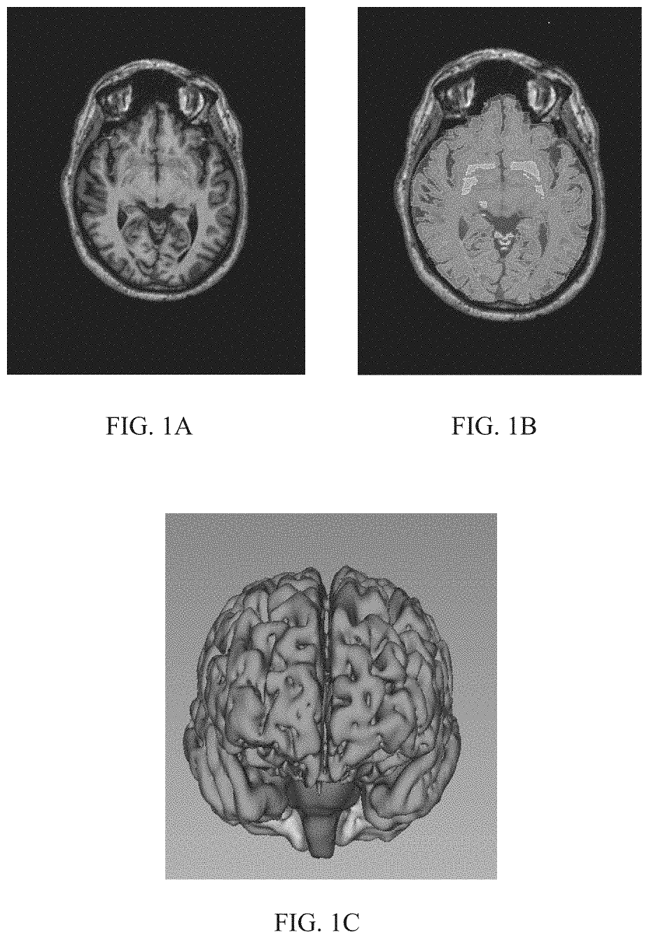

Turning to FIGS. 1A-1C, converting the T1 MRI image (FIG. 1A) to a segmented mask (FIG. 1B) and a volumetric model (FIG. 1C) using deep learning models is demonstrated. FIG. 1A depicts T1 MRI on an axial plane. In FIG. 1B, a deep learning-based segmentation mask is applied to the 2-D image of FIG. 1A. Finally, FIG. 1C shows a 3D structure of the anatomy from a number of MRI images. As seen in the 3D image of FIG. 1C, the volumetric model enhances the visualization compared to the tradition 2D MRI images. Further, because the volumetric image is generated from many MRI images taken throughout the organ, the interior of the image will also display the unique features of the patient's anatomy including the targets of the surgical intervention such as anatomical abnormalities or objects such as tumors.

Using custom hand gestures and virtual surgical equipment, surgeons can simulate the surgery on the 3D anatomical AR model based on the preplanning data. Simulation of surgical procedures has a higher accuracy impact on the intraoperative procedures.

During the intraoperative stage, the 3D model of the targeted anatomical part is superimposed on the surgical region of interest at a 1:1 ratio using the augmented reality glass. The superimposed augmented reality 3D model on the surgical region of interest unfolds the view of the inner structure of the anatomy which is not visible with the naked eyes. Inner structural details of the anatomy with the guided view of augmented reality reveal more information regarding the patient's anatomy.

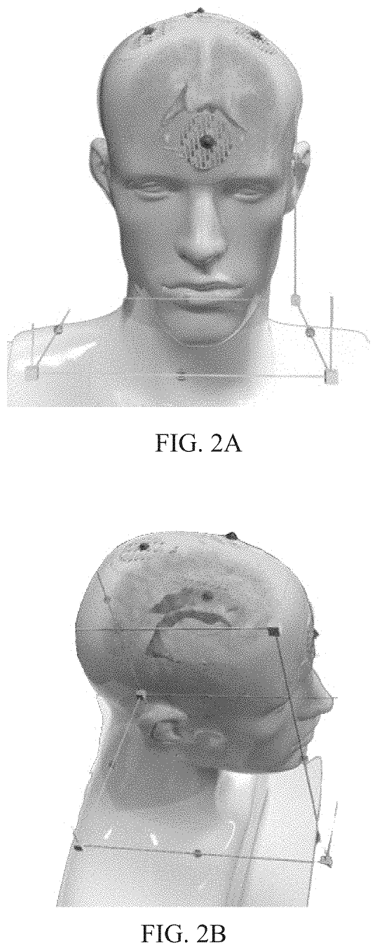

The system 100 is tested by targeting a neurosurgical procedure on a dummy model. As shown in FIGS. 2A-2B, the implementation of the superimposition-based AR surgical tracking, a 3D reconstructed ventricle is successfully and accurately superimposed on the dummy head from all viewing angels. The superimposition of 3D anatomical model onto a dummy allows surgeons or any participants in medical planning to see the inner structures of brain.

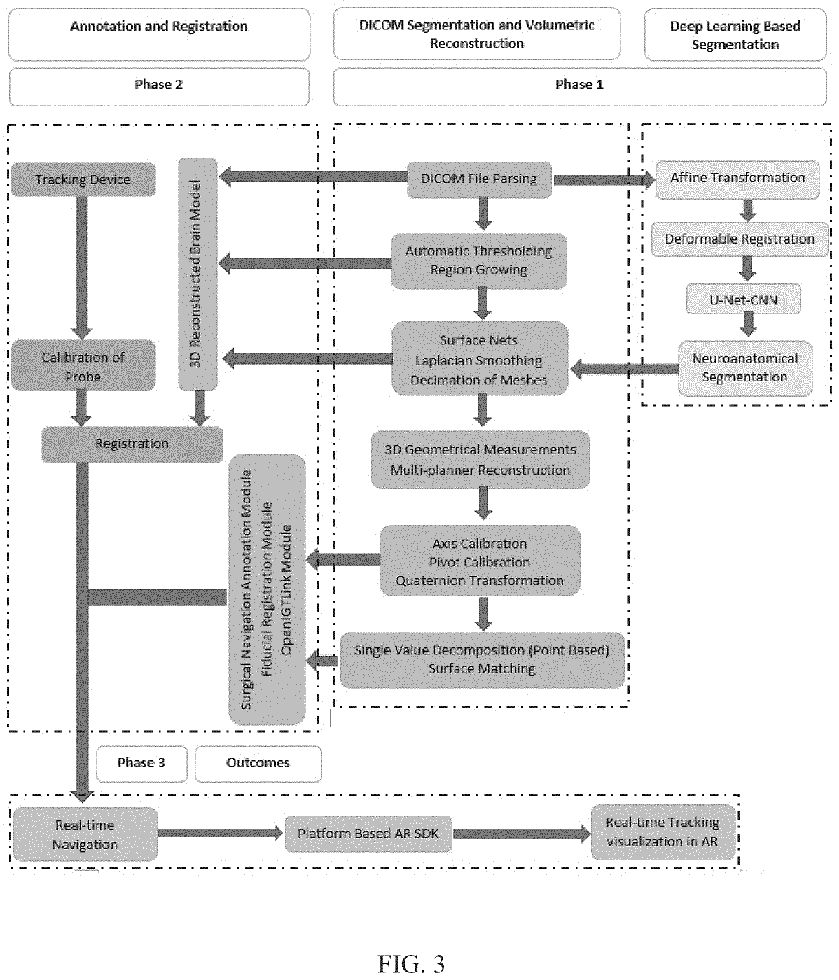

From the MRI data processing to the intraoperative real-time surgical tracking system with the patient specific anatomical 3D AR models, a pipeline of refined algorithms is provided. As shown in FIG. 3, the workflow of the actions from MRI data to intraoperative 3D anatomical AR-based surgical tracking is exhibited. As seen in FIG. 3, Phase 1 involves a deep learning based segmentation and DICOM segmentation and volumetric reconstruction. This phase is used with the annotation and registration phase 2 involving a physical tracking device used in a surgical procedure. Clinical imaging data is typically provided in a DICOM format (Digital Imaging and Communications in Medicine). DICOM is a tag-based format where objects in a file are encapsulated in a tag that describes the object and its size. Affine transformation involves linear mapping that preserves points, lines, and planes, and can correct distortions that result from images that were takes with less-than-ideal image capture perspectives. These transformed images undergo deformable registration and are applied to a U-net convolutional network for segmentation of images. It is a sliding window convolutional network that has been used for biomedical image segmentation. The process image data undergoes neuroanatomical segmentation and is routed to be processed with surface nets, Laplacian smoothing and decimation of meshes after which it can be used in the formation of a 3D reconstructed anatomical image—in this case, in FIG. 3, a 3D reconstructed brain model.

The 3D brain model is used in combination with a tracking device (following probe calibration) to undergo registration in Phase 2. In parallel, the neuroanatomical segmented data undergoes 3D geometrical measurements and multi-planar reconstruction. MPR converts data from an imaging modality, acquired in an axial plane, into another plane. This converted data undergoes axis calibration, pivot calibration, and quaternion transformation. This information is applied in Phase 2 to create a surgical navigation annotation module, along with a fiducial registration module and, for the particular optical system used as an exemplary embodiment, an Open IGT Link module. Additionally, the transformed data undergoes single value decomposition (point based) surface matching, which is also used in the various modules described above.

Finally in Phase 3, real-time navigation applies the processed data from Phase 1 and Phase 2 in order to provide real-time tracking visualization in Augmented Reality.

### INDUSTRIAL APPLICABILITY

The present invention leverages visualization techniques using the current AR technology combined with real time tracking. Currently, there is no AR-based surgical tracking technique or product available on the market. Most of the AR based surgical navigation from multiple research outcomes can be classified into two areas: first, using head mounted display (HMD) to superimpose the augmented anatomical structure onto the real patient without any tracking; second, displaying the tracking data of surgical equipment (probe) onto an anatomical model. Few of the common approaches in all the on-going research focuses on the visualization technique and enhancement technique instead of providing the spatial information to visualize the 3D structure of the anatomy, tracking efficiency, data processing and user end platform.

The following is some key distinctive advantages of the present invention compared with the currently available commercial products and research:
1. MRI data are segmented and processed to generate the volumetric structure of the anatomy without human intervention. A manual segmentation of MRI set used in the conventional system and method is very time-consuming and labor-intensive. The present invention improves the efficiency of segmentation and volume rendering by using a deep-learning-based segmentation technique through a deep neural network.
2. The present invention resolves the problem of incorporating AR techniques in displaying a 3D model preserving the spatial information after transforming from one coordinate to another.
3. To improve the efficiency of tracking, a combined optical and electromagnetic tracking system is provided and the data generated therefrom is validated through an Unscented Kalman Filter which is cascaded by a deep neural network for data forecast before imposing. Unscented Kalman filter is very robust in comparing and calculating the coordinate data generated from the combined optical and electromagnetic tracking system. Using the deep neural network along with the Unscented Kalman filter to predict the path of movement of surgical equipment improves accuracy of the prediction. The network is trained based on the collected data of surgeons' hand movement. One advantage of cascading the Kalman filter with deep neural network is the accuracy level and successful path-prediction which reduces the risk, especially in complex organ surgeries such as Spine Surgery.
4. One of the major drawbacks of the existing surgical navigation system is the usability cost. Normally, tracking data cannot be visualized without using proprietary software or device. However, the present invention provides an application platform independent of these which can be provided with mobile support which provides easy accessibility to surgeons and medical professionals, and thereby reducing their overall operational cost.
5. The augmented reality 3D anatomy based on patient-specific magnetic resonance imaging (MRI) images can apply to surgeon training in medical schools as an advance over traditional carcass-based teaching, due to its accessibility, mobility and usability.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the specification, and following claims.

1. An MRI-based surgical navigation method of providing a personalized augmented reality of targeted internal organs of a subject with real-time intraoperative tracking, comprising:
obtaining a plurality of two-dimensional magnetic resonance imaging images of targeted internal organs by a magnetic resonance imaging device;
segmenting one or more of the two-dimensional magnetic resonance imaging images into a plurality of segmented data and recombining thereof to generate a three-dimensional volumetric model of the targeted internal organs;
providing an augmented reality-based three-dimensional simulation to obtain an augmented reality-based three-dimensional simulation model including anatomical features and spatial information of the targeted internal organs, and overlaying thereof with the three-dimensional volumetric model of the targeted internal organs while gaining a real-time feedback of one or more surgical operations carried out on the targeted internal organs;
processing data of the anatomical features and spatial information of the targeted internal organs to generate a plurality of robust and accurate navigation coordinates; and
outputting the plurality of robust and accurate navigation coordinates to an augmented reality-based three-dimensional simulation and real-time anatomical model capture and display device for assisting medical practitioners of the one or more surgical operations to visualize at least a surgical path and specific anatomical features of an individual receiving said surgical operations;
wherein the plurality of robust and accurate navigation coordinates is calculated and generated by a combined optical and electromagnetic tracking system with the following steps:
marking optical markers on the targeted internal organs based on fiducials marker attached to the surgical area;
tracking the optical markers by the combined optical and electromagnetic tracking system and generating a set of tracking data; and
feeding the set of tracking data to a filter that transforms the tracking data through a non-linear function to generate the coordinate.

2. The method of claim 1, wherein the segmenting and recombining are carried out by a deep neural network to generate the three-dimensional volumetric model of the targeted internal organs with unique identification of non-specific and specific anatomical features.

3. The method of claim 1, wherein the augmented reality-based three-dimensional simulation and real-time anatomical model capture and display device collects at least one body appearance feature of the subject from a user's direction of view, wherein the at least one body appearance feature is in registration with a human appearance characteristics image database.

4. The method of claim 3, wherein the plurality of robust and accurate navigation coordinates is calculated based on the plurality of two-dimensional magnetic resonance imaging images and the at least one body appearance feature of the subject.

5. The method of claim 1, wherein the filter is an unscented Kalman filter for transforming the data points through the non-linear function in order to obtain a deep learning-based data forecast model.

6. The method of claim 5, wherein the unscented Kalman filter is cascaded with a deep neural network for predicting the surgical path of the medical instrument in the targeted internal organs.

7. A surgical navigating system for providing patient-specific and surgical environment-specific pre-operative planning and intraoperative navigation, comprising:
a magnetic resonance imaging (MRI) device for capturing a plurality of two-dimensional magnetic resonance imaging images of targeted internal organs;
a deep neural network for segmenting the plurality of the two-dimensional magnetic resonance imaging images of the targeted internal organs to obtain segmented data of the two-dimensional magnetic resonance imaging images and recombining the segmented data to generate a three-dimensional volumetric model of the targeted internal organs;
a combined optical and electromagnetic tracking system for acquiring data of optical markers' location at the targeted internal organs and transforming the data points through a non-linear function; and
an augmented reality-based three-dimensional simulation and real-time anatomical model capture and display device for creating a three-dimensional anatomical simulation model during a simulated surgical operation based on the three-dimensional volumetric shape of the targeted human body part, collecting at least one body appearance features, gathering real-time feedback of one or more surgical operations, overlaying the three-dimensional volumetric shape of the targeted human body part with the three-dimensional anatomical simulation model, displaying a predicted surgical path of medical instrument obtained during a surgery simulation process including navigation coordinates of medical instruments and specific anatomical features of an individual receiving the surgical operation.

8. The system of claim 7, wherein the augmented reality-based three-dimensional simulation and real-time anatomical model capture and display device collects at least one body appearance feature of the subject from a user's direction of view, wherein the at least one body appearance feature is in registration with a human appearance characteristics image database.

9. The system of claim 7, wherein the combined optical and electromagnetic tracking system processes data of the anatomical features and spatial information of the targeted internal organs to generate a plurality of robust and accurate navigation coordinates.

10. The system of claim 9, wherein the plurality of robust and accurate navigation coordinates is calculated based on the plurality of two-dimensional magnetic resonance imaging images and the at least one body appearance feature of the subject.

11. The system of claim 7, wherein the combined optical and electromagnetic tracking system comprises a filter for transforming the data points through the non-linear function.

12. The system of claim 11, wherein the filter is an unscented Kalman filter cascaded with the deep neural network for predicting the surgical path of the medical instrument in the targeted internal organs.
